(19) United States
(12) Patent Application Publication
(10) Pub. No.: US 2020/0312444 A1
(43) Pub. Date: Oct. 1, 2020

(54) SYSTEMS AND METHODS FOR METRIC-BASED GRAPHICAL USER INTERFACES IN HEALTHCARE OPERATIONS

(71) Applicant: TeleTracking Technologies, Inc., Pittsburgh, PA (US)

(72) Inventor: William Griffith, Lighthouse Point, FL (US)

(73) Assignee: TeleTracking Technologies, Inc., Pittsburgh, PA (US)

(21) Appl. No.: 16/831,091

(22) Filed: Mar. 26, 2020

Related U.S. Application Data
(60) Provisional application No. 62/824,989, filed on Mar. 27, 2019.

(51) Int. Cl. G16H 40/20; G06Q 40/02

(52) U.S. Cl. CPC G16H 40/20; G06Q 40/02

(57) ABSTRACT

The present disclosure relates to systems and methods for system for automatically generating healthcare metrics. In one implementation, such a system may comprise at least one memory storing instructions; and at least one processor configured to execute the instructions to: retrieve, from one or more networked computer systems, one or more distributions, each distribution associated with a healthcare operating variable, the one or more networked computer systems collating the one or more distributions based on real-time patient-by-patient input; retrieve, from one or more financial systems, budgetary information classified as related to the one or more associated healthcare operating variables; calculate, using the budgetary information, one or more conversion factors from the one or more associated healthcare operating variables to one or more financial variables; generate the one or more financial variables using the one or more conversion factors; and output a report including the one or more financial variables.

### CROSS-REFERENCE TO RELATED APPLICATIONS

This application claims priority to U.S. Provisional Patent App. No. 62/824,989, filed on Mar. 27, 2019, which is incorporated herein by reference in its entirety.

### TECHNICAL FIELD

The present disclosure relates generally to the fields of graphical user interfaces and networked computer systems. More specifically, and without limitation, this disclosure relates to systems and methods for automatically generating graphical user interfaces displaying healthcare facility operation metrics, using networked healthcare computer systems.

### BACKGROUND

Traditional mechanisms for healthcare operation metrics focus on average statistics, such as average discharge time, average discharge compliance, or the like. However, such averages often obscure problems with distributions of operational statistics on a patient-by-patient level. Accordingly, some healthcare institutions have introduced systems with detailed patient-by-patient data intake to produce accurate distributions rather than overall averages.

However, patient-by-patient data is not suitable for consumption by traditional management systems. Moreover, even aggregating this data into averages, medians, or the like is not useful for traditional management systems. Accordingly, existing systems may use subjective and manual conversion of patient statistics into financial statistics. However, such conversion is time-consuming and difficult to undertake, and it uses unreliable financial indicators. Performing the necessary calculations at the scale required to evaluate the performance of an entire facility is often infeasible or impossible using manual or traditional techniques, due to large amounts of data, and the fast-paced nature of healthcare facility operations. Thus, traditional techniques are unable to effectively keep up with the rapid changes in facility operations.

Moreover, traditional techniques involve manual formatting of the data formatted for consumption by audiences, often using inconsistent and inaccurate manual and subjective methodologies, which may lead to errors or inaccuracies in results produced.

In view of the drawbacks and deficiencies discussed above, improved systems and methods for generating healthcare operation reports are desired.

### SUMMARY

Systems and methods are disclosed for automating the aggregation of operational metrics in healthcare facilities, from discrete networked computer systems or different subsystems of a healthcare facility system. Disclosed embodiments also replace subjective analyses of traditional techniques with automatic and rule-based statistical analyses of the aggregated healthcare operation data, using particular rules and mechanisms disclosed herein. Based on the statistical analyses, Some embodiments of disclosed systems also use arrangements of sensors across one or more healthcare facilities in combination with particular database structures to allow for such aggregation and analysis automation.

In addition, the provided systems and methods may automatically format and construct summaries of the generated metrics based on an analysis of the generated metrics and application of rule sets that correlate stored summary statements to certain metric ranges. For example, the provided systems and methods may generate metric reports including automatically formatted visual representations of the metrics coupled with summary statements and recommendations automatically matched to the metrics using one or more rules. Accordingly, the disclosed embodiments may improve users' experiences with healthcare metric systems.

Some disclosed embodiments describe a system for automatically generating healthcare metrics, which may comprise at least one memory storing instructions and at least one processor configured to execute instructions. The instructions may include instructions to retrieve, from one or more networked computer systems, one or more distributions, each distribution associated with a healthcare operating variable, the one or more networked computer systems collating the one or more distributions based on real-time patient-by-patient input; retrieve, from one or more financial systems, budgetary information classified as related to the one or more associated healthcare operating variables; calculate, using the budgetary information, one or more conversion factors from the one or more associated healthcare operating variables to one or more financial variables; generate the one or more financial variables using the one or more conversion factors; and output a report including the one or more financial variables.

In accordance with further embodiments, the at least one processor is further configured to prioritize the one or more financial variables.

In accordance with further embodiments, the at least one processor is further configured to prioritize the one or more associated healthcare operating variables based on the prioritization of the one or more financial variables, and wherein the report further includes the one or more associated healthcare operating variables.

In accordance with further embodiments, the at least one processor is configured to prioritize the one or more financial variables according to one or more settings input by a user.

In accordance with further embodiments, the at least one processor is configured to automatically prioritize the one or more financial variables according to a projected impact on at least one of revenue or cost.

In accordance with further embodiments, the at least one processor is configured to output the report on a periodic basis, and re-prioritize the one or more financial variables each time a report is output.

Some disclosed embodiments describe a system for automatically summarizing a generated healthcare metric report, which may comprise at least one memory storing instructions and at least one processor configured to execute the instructions. The instructions may include instructions to receive the healthcare metric report including one or more healthcare operating variables compared to one or more associated baselines and one more financial variables related to the one or more healthcare operating variables; using a first series of logic rules applied to the one or more healthcare operating variables and associated one or more baselines, determine at least one first summary sentence; using a second series of logic rules applied to the one or more financial variables, determine at least one second summary sentence; generate an executive summary including the at least one first summary sentence and the at least one second summary sentence; and append the generated executive summary to the received report and outputting the report after appending.

In accordance with further embodiments, the at least one processor is further configured to prioritize the one or more financial variables, and wherein determining the at least one second summary sentence is based on the prioritization.

In accordance with further embodiments, the at least one processor is further configured to prioritize the one or more healthcare operating variables based on the prioritization of the one or more financial variables, and wherein determining the at least one first summary sentence is based on the prioritization of the one or more healthcare operating variables.

In accordance with further embodiments, the at least one processor is configured to prioritize the one or more financial variables according to one or more settings input by a user.

In accordance with further embodiments, the at least one processor is configured to automatically prioritize the one or more financial variables according to a projected impact on at least one of revenue or cost.

In accordance with further embodiments, the at least one processor is configured to generate the executive summary on a periodic basis, and re-prioritize the one or more financial variables each time an executive summary is generated.

Some disclosed embodiments describe a method for automatically generating healthcare metrics, comprising: retrieving, from one or more networked computer systems, one or more distributions, each distribution associated with a healthcare operating variable, the one or more networked computer systems collating the one or more distributions based on real-time patient-by-patient input; retrieving, from one or more financial systems, budgetary information classified as related to the one or more associated healthcare operating variables; calculating, using the budgetary information, one or more conversion factors from the one or more associated healthcare operating variables to one or more financial variables; generating the one or more financial variables using the one or more conversion factors; and outputting a report including the one or more financial variables.

In accordance with further embodiments, the method further comprises prioritizing the one or more financial variables.

In accordance with further embodiments, the method further comprises prioritizing the one or more associated healthcare operating variables based on the prioritization of the one or more financial variables, and wherein the report further includes the one or more associated healthcare operating variables.

In accordance with further embodiments, the method further comprises prioritizing the one or more financial variables according to one or more settings input by a user.

In accordance with further embodiments, the method further comprises automatically prioritizing the one or more financial variables according to a projected impact on at least one of revenue or cost.

In accordance with further embodiments, the report is output on a periodic basis, and the method further comprises re-prioritizing the one or more financial variables each time a report is output.

In some embodiments, the present disclose describes non-transitory, computer-readable media for causing one or more processors to execute methods and techniques disclosed herein.

It is to be understood that the foregoing general description and the following detailed description are example and explanatory only, and are not restrictive of the disclosed embodiments.

### BRIEF DESCRIPTION OF THE DRAWINGS

The patent or application file contains at least one drawing executed in color. Copies of this patent or patent application publication with color drawing(s) will be provided by the United States Patent and Trademark Office upon request and payment of the necessary fee.

The accompanying drawings, which comprise a part of this specification, illustrate several embodiments and, together with the description, serve to explain the principles disclosed herein. In the drawings:

FIG. 1 is a block diagram of a system for generating a healthcare metric report and graphical user interface, according to an exemplary embodiment.

FIG. 2 is a block diagram of a system for automatically generating a healthcare metric report, according to an exemplary embodiment.

FIG. 3A is a graphical representation of an example graphical user interface with visual representations of healthcare operating metrics, according to an exemplary embodiment.

FIG. 3B is a graphical representation of an example of a graphical user interface with visual representations of healthcare facility metrics, according to an exemplary embodiment.

FIG. 3C is a graphical representation of an automatically generated executive summary graphical user interface, according to an exemplary embodiment.

FIG. 3D is another graphical representation of an exemplary automatically generated executive summary graphical user interface, according to an exemplary embodiment.

FIG. 4 is a flowchart of an exemplary method for automatically generating a healthcare metric report, according to an exemplary embodiment.

FIG. 5 is a flowchart of an example method for automatically summarizing a generated healthcare metric report, according to an example embodiment.

FIG. 6 is a block diagram of an exemplary server with which the systems, methods, and apparatuses of the disclosed embodiments may be implemented.

FIG. 7 is a block diagram of an example of a sensor device for collecting patient data for inclusion in systems and processes disclosed herein.

### DETAILED DESCRIPTION

The disclosed embodiments relate to systems and methods for automatically generating and summarizing healthcare metric reports. Disclosed embodiments may be implemented using general-purpose computer hardware programmed with special purpose software to perform functions disclosed herein. Alternatively, a special-purpose computer may be built according to embodiments of the present disclosure using suitable logic elements and specialized hardware.

Advantageously, disclosed embodiments may automatically visualize healthcare metrics based on patient-specific data, using distributed computing systems for compiling performance data used for benchmarking, dynamic threshold generation and updating, and for evaluating individual facilities or groups of facilities. Moreover, disclosed embodiments may improve upon prior subjective manual techniques and systems that lacked sufficient computing logic to effectively generate healthcare metric reports, by analyzing data using particular rule sets, and consistent and dynamic thresholds.

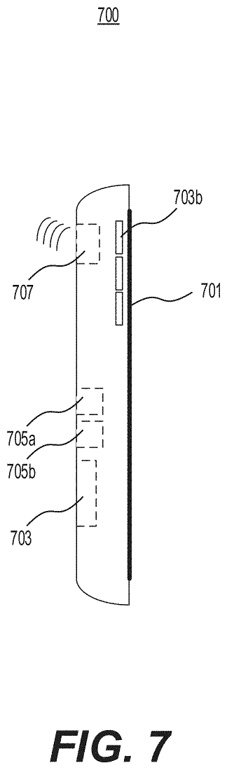

According to an aspect of the present disclosure, one or more servers or other computing devices may retrieve, from one or more networked computer systems, one or more distributions. For example, the one or more networked computer systems may be associated with and receive patient-level statistics from one or more devices within a healthcare system. For example, the one or more networked computer systems may receive waiting times, intake times, bed assignment times, and other patient-level statistics from an intake device (such as a laptop computer, a desktop computer, a mobile device such as device 700 of FIG. 7, or the like). The intake device may be associated with an emergency room, a preparation room for surgery, a waiting room for patients, or the like. Additionally or alternatively, the one or more networked computer systems may receive transport times, hold times, delays, or the like from a device (such as a laptop computer, a desktop computer, a mobile device such as device 700 of FIG. 7, or the like) associated with an ambulance, an internal transport crew, or the like. Additionally or alternatively, the one or more networked computer systems may receive imaging times, wait times, delays, correction rates, or the like from a medical imaging device (such as a magnetic resonance imaging (MRI) machine, a computed tomography (CT) scanner, or the like). A device may also receive patient information during treatment, such as tests and/or procedures conducted in a department associated with radiology, Cath, IR, Surgery, GI, and the like. For example, a device within a surgery department may receive manual or automatic inputs related to a start time and end time of a surgery of a patient, which may allow it to compute a time of operation. Such computations can become part of patient-level statistics (e.g., from aggregated data from multiple patient surgeries). Additionally or alternatively, the one or more networked computer systems may receive wait times, discharge times, bed turnaround times, and other patient-level statistics from a discharge device (such as a laptop computer, a desktop computer, a mobile device such as device 700 of FIG. 7, or the like). The discharge device may be associated with an emergency room, a surgery room, a post-operative recovery room, or the like.

In some embodiments, each distribution may be associated with a healthcare operating variable, such as any of the operating variables described above. Moreover, as explained above, the one or more networked computer systems may collate the one or more distributions based on real-time patient-by-patient input (e.g., received by at least one sensor, as further discussed below). For example, whenever a patient is admitted, an intake device (such as a laptop computer, a desktop computer, a mobile device such as device 700 of FIG. 7, or the like associated with an emergency room, a preparation room for surgery, a waiting room for patients, or the like) may send data including a waiting time, intake time, and/or other patient admittance data. Such data may be sent in response to an input received at an intake device (e.g., through an order entry into EMR). Inputs may be manual or automatic (i.e., sent in response to a sensor detecting the admitted patient, a scanning device scanning a patient wristband, etc.). Similarly, whenever a patient is assigned a bed, the intake device may send a waiting time, a bed assignment time, or the like upon assignment. In another example, whenever a patient has one or more medical images captured, the medical imaging device (such as an MRI machine, a CT scanner, or the like) may send an imaging time, a waiting time, or the like. In yet another example, whenever a patient is discharged, a discharge device (such as a laptop computer, a desktop computer, a mobile device such as device 700 of FIG. 7, or the like associated with an emergency room, a surgery room, a post-operative recovery room, or the like) may send a waiting time, a discharge time, a bed turnaround time, or the like. In another example, whenever a surgery begins or ends, a device (such as a laptop computer, a desktop computer, a mobile device such as device 700 of FIG. 7) associated with the surgery room and/or the surgeon may send a waiting time, a surgery time, an operation room turnaround time, or the like. In yet another example, whenever a checkout or consultation or other doctor visit begins or ends, a device (such as a laptop computer, a desktop computer, a mobile device such as device 700 of FIG. 7) associated with the room and/or the doctor may send a waiting time, an appointment length, a room turnaround time, or the like.

Any of the statistics described above may be sent over one or more computer networks, such as the Internet, a local area network (LAN), or the like, and may be sent using WiFi, 4G, Ethernet, or the like. In some embodiments, to retain security, the statistic(s) may be sent over a private network (such as a LAN) and/or may be encrypted (e.g., using an Advanced Encryption Standard (AES)). In embodiments where the statistic(s) is (are) encrypted, the receiving server may decrypt the request using a private key. In embodiments where the receiving server forwards the update to a different server for storage and indexing, the receiving server may forward the encrypted update without decrypting the update first or may decrypt the update and forward the decrypted update. In embodiments where the receiving server decrypts the update, the decrypted update may be sent along a private channel (such as a private network) to the different server.

The one or more servers or other computing devices may further retrieve, from one or more financial systems, budgetary information classified as related to the one or more associated healthcare operating variables. For example, the one or more servers may receive daily revenue, daily patient numbers, daily bed numbers, daily operating costs, or the like in order to determine a net per patient contribution margin, an operating cost per bed per day, a cost per hold per hour, average cost of healthcare-associated infection, or the like. The one or more financial systems may send the statistics similarly to the one or more servers described above.

The one or more servers or other computing devices may calculate, using the budgetary information, one or more conversion factors from the one or more associated healthcare operating variables to one or more financial variables. For example, the one or more servers may determine revenue per patient using revenue statistics from the one or more financial systems and patient statistics from the one or more networked computer systems. In another example, the one or more servers may determine operating cost per patient using revenue statistics from the one or more financial systems and patient statistics from the one or more networked computer systems.

In some embodiments, the one or more servers or other computer devices may determine a conversion factor based on examining at least one patient attribute. For example, a server may identify a particular attribute associated with a patient (e.g., admitted for trauma surgery) and determine a relationship between the attribute and a financial variable (e.g., a patient admitted for trauma surgery increases the total cost of patient stay by 65%). In some embodiments, patient attributes may be stored in and retrieved from one or more databases (e.g., patient statistics database 103, discussed further below). In some embodiments, a relationship may be determined by aggregating data associated with multiple patient attributes, which may be associated with multiple patients (e.g., multiple patient admitted for trauma surgery). A relationship between a patient attribute and a financial variable may be used to influence, improve, generate, etc. one or more conversion factors. In some embodiments, a patient attribute and/or relationship may be included as part of patient-by-patient input.

In some embodiments, the one or more servers or other computing devices may project changes in financial statistics based on changes in patient statistics. For example, the one or more servers may project additional revenue from additional admissions and/or reduced operating costs if the average length of stay decreases. In another example, the one or more servers may project additional revenue from additional admissions and/or reduced operating costs if the average bed turnaround time decreases. In yet another example, the one or more servers may project additional revenue from additional admissions and/or reduced operating costs if the average intake time decreases. In yet another example, the one or more servers may project an expected reduction in cost per discharge if the average discharge time decreases. In yet another example, the one or more servers may project additional revenue from additional admissions and/or reduced operating costs if the average transport time and/or delay decreases. Accordingly, the one or more servers may generate the one or more financial variables using the one or more conversion factors.

In some embodiments, one or more conversion factors may be determined according to a machine-learning process. For example, a device (e.g., content management server 102) may determine, such as through using operating data and financial variables, an impact that the operating data has on a financial variable. Based on the determined impact, the device may produce a recommendation (e.g., a recommendation to change a variable leading to a change in operating data, a recommendation to change a conversion factor, etc.) using current conversion factors. In some embodiments, for example where a change is implemented (e.g., based on a recommendation), the device may perform further analysis to determine a new impact based on the implemented change. Thus, the device may, through iterative analysis and intervening changes, learn relationships, correlations, etc. between operating data and financial variables, which may be based on implemented changes (e.g., reducing a patient wait time). In some embodiments, a machine-learning process may be performed using a model with at least one parameter, which may be based on a user-set policy. For example, a model may have parameters associated with priorities of a hospital (or other institution), such a relative priority between two operating variables, between an operating variable and a financial variable, etc. In some embodiments, a parameterized model may be used to configure one or more conversion factors.

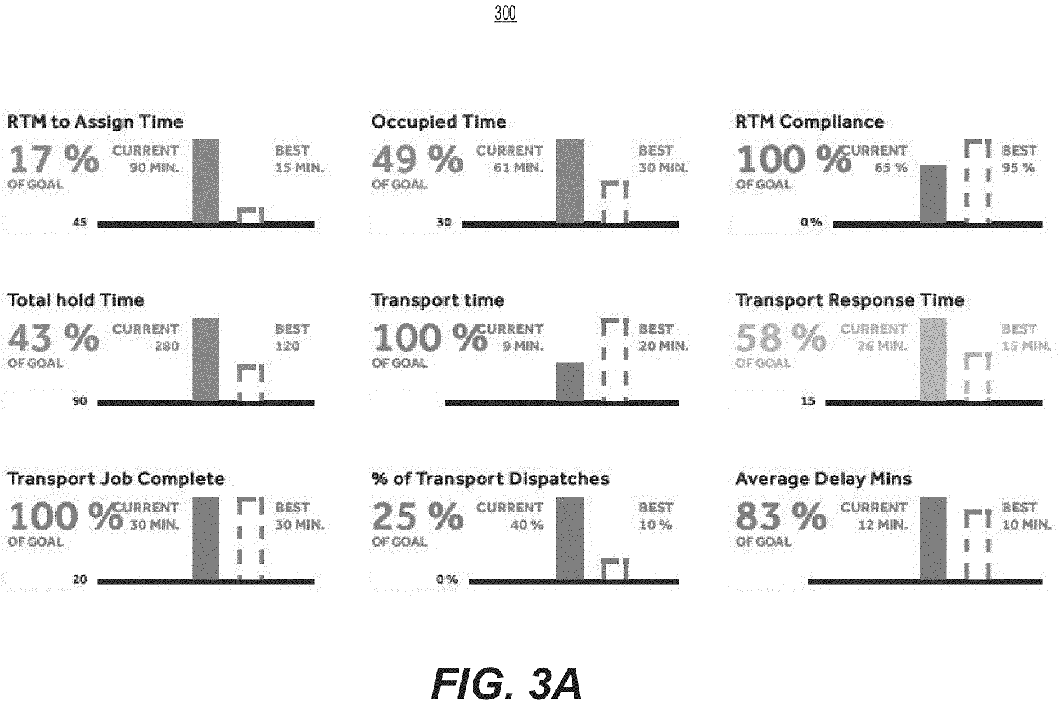

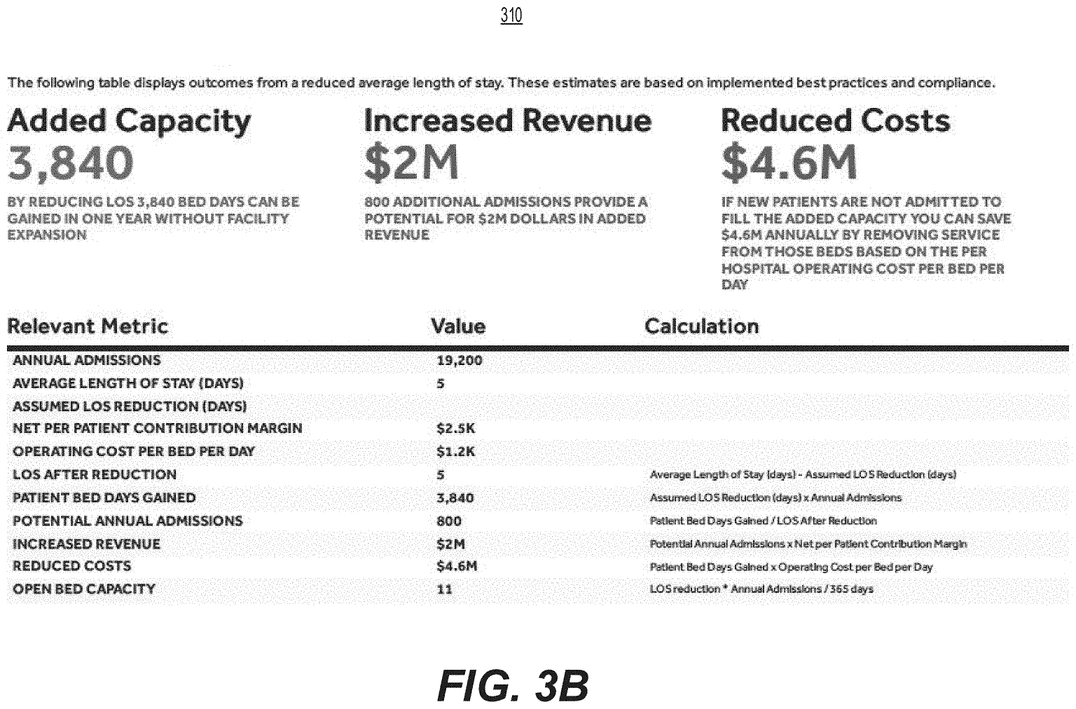

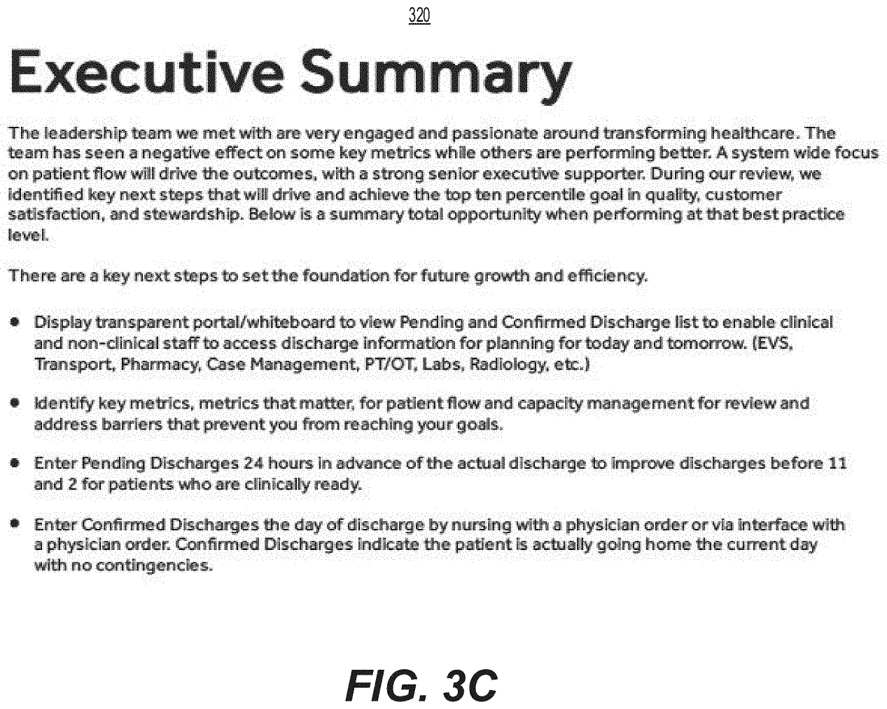

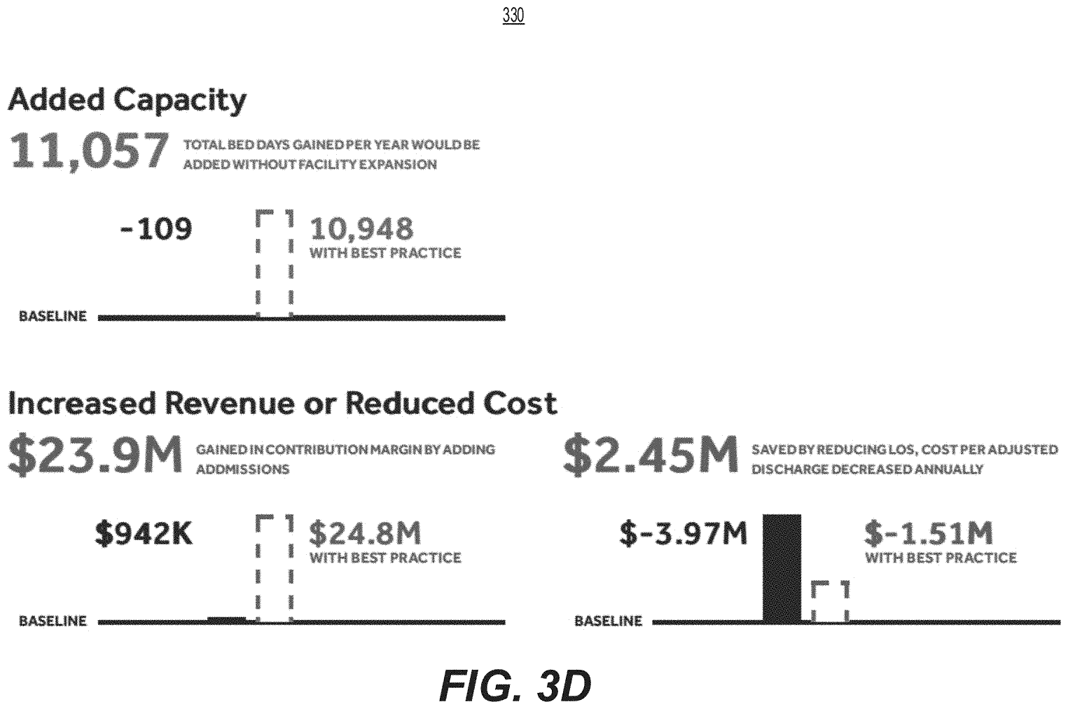

The one or more servers or other computing devices may output a report including the one or more financial variables. For example, the report may include one or more visual indicators of the operating variables (as depicted in FIG. 3A) and/or one or more visual indicators of the financial variables (as depicted in FIG. 3B). For example, although depicted as bar graphs in FIGS. 3A and 3B, additional or alternative, visual indicators may be used, such as line charts, pie charts, or the like. Additionally or alternatively, the report may include an executive summary (as depicted in FIG. 3C) based on the operating variables and/or the financial variables. The executive summary may be automatically generated as described below.

In some embodiments, one or more servers or other computing devices may receive the healthcare metric report. The report may include one or more healthcare operating variables (such as a waiting time for intake, an intake time, a waiting time for bed assignment, a bed assignment time, a waiting time for imaging, an imaging time, a waiting time for discharge, a discharge time, a bed turnaround time, a waiting time for surgery, a surgery time, an operation room turnaround time, a waiting time for a doctor, an appointment length, a room turnaround time, or the like) compared to one or more associated baselines. For example, the baselines may be determined using the operating variables (and, accordingly, may be an average, median, or other statistical measure), may be based on industry-wide measurements (e.g., an average, median, or the like of all other healthcare companies, healthcare companies classified in the same geographic area, size category, or the like as the company associated with the report), or may comprise a goal, which may be defined by a user locally (e.g., by an input at content management server 102), or by a remote system (e.g., an industry best practice measurement defined at a source external to content management server 102). Additionally or alternatively, the report may include one more financial variables (such as net per patient contribution margin, an operating cost per bed per day, a cost per hold per hour, average cost of healthcare-associated infection, or the like) related to the one or more healthcare operating variables. Accordingly, the report may have been generated as described above.

Using a first series of logic rules applied to the one or more healthcare operating variables and associated one or more baselines, the one or more servers or other computing devices may determine at least one first summary sentence. For example, the logic rules may select the one or more operating variables having a greatest discrepancy with associated baselines. Based on the identity of such variables, the logic rules may select one or more predetermined first summary sentences. In some embodiments, the logic rules may select from a ranked list of first summary sentences based on current policies of the healthcare company associated with the received report. For example, a policy may have a corresponding assessment model with at least one parameter, which may be based on a user-set policy, which may be configured at a content management server 102 or at another computing device. For example, a first summary sentence may comprise “Display transparent portal/whiteboard to view Pending and Confirmed Discharge list,” but the logic rules may select a different first summary sentence (e.g., “Enter Pending Discharges 24 hours in advance of the actual discharge”) if the healthcare company already implements a transparent portal/whiteboard.

In another example, a first summary sentence may comprise “Utilize notifications to inform sending/receiving areas that a job has been created/cancelled/delayed, or that a transporter is on the way to collect/deliver a patient,” but the logic rules may select a different first summary sentence (e.g., “Centralize the Transport Department to deliver efficient and dependable Transport service using an efficiency focused dispatch set”) if the healthcare company already implements a system with such notifications. In yet another example, a first summary sentence may comprise “Implement a discharge clean team without competing priorities to improve the bed turn times,” but the logic rules may select a different first summary sentence (e.g., “Utilize transport when the patient is leaving their room for discharge, to ensure real time notification of the dirty bed”) if the healthcare company already implements a discharge clean team.

Additionally or alternatively, the logic rules may be employed by one or more of the processors disclosed herein to select a number of first summary sentences based on a size of a discrepancy between the determined operating variable and the associated baseline value for the variable. For example, in the example of FIG. 3C, a plurality of first summary sentences based on one or more discharge variables have been selected.

In some embodiments, the logic rules may prioritize one or more financial variables based on projected revenue increase and/or cost decrease. For example, the logic rules may be employed by one or more of the processors disclosed herein to select one or more operating variables for which a corresponding change in revenue and/or cost is largest and use the selected operating variable(s) to generate the at least one first summary sentence. Additionally or alternatively, one or more user settings may be retrieved and used by the logic rules to select the one or more operating variables. For example, the one or more user settings may include a roadmap or other indicator of a progression of operating variables to prioritize. A roadmap may include dynamic sequence of goals, which may be updated to reflect progress and/or regress according to an assessment model. In such an example, the logic rules may select the next operating variable(s) on the roadmap to use for generating the at least one summary sentence. In such embodiments, the logic rules may progress backwards along the roadmap to ensure compliance with previous operating variables such that, if an operating variable that had previously progressed in a desired direction (e.g., above a progression threshold) reversed in the opposite direction (e.g., below a backtracking threshold), the logic rules may select the operating variable that reversed to use for generating the at least one summary sentence. In another example, the one or more user settings may include operating variables to select for generating the at least one summary sentence. Prioritization of the one or more financial variables may track prioritization of one or more operating variables or vice versa.

Moreover, although described as above, in some embodiments, the at least one first summary sentence may include one or more portions into which the logic rules may insert variables particular to the healthcare system. For example, the logic rules may select “Discharge window of [##] minutes is causing [##] hours of Length of Stay” and fill the indicated portions (labeled [##]) with values of appropriate operating variables from the corresponding healthcare system. In another example, the logic rules may select “EVS turn time of [##] minutes is adding [##] hours of Dead Bed Time” and fill the indicated portions (labeled [##]) with values of appropriate operating variables from the corresponding healthcare system.

Using a second series of logic rules applied to the one or more financial variables, the one or more servers or other computing devices may further determine at least one second summary sentence. For example, the logic rules may select the one or more financial variables most likely to be affected by (and/or having a largest change based on) a change in operating variables. Based on the magnitude of the change in financial variables, the logic rules may generate one or more second summary sentences. For example, in the example of FIG. 3D, a second summary sentence indicating that “$[##]M [will be] saved by reducing LOS [length of stay], cost per adjusted discharge decreased annually” is selected. As explained above, the logic rules may fill the indicated portions (labeled [##]) with values of appropriate financial variables from the corresponding healthcare system. In yet another example, a second summary sentence may comprise “[##] additional patients will be admitted which accounts for $[##]M in potential added revenue” with the logic rules filling the indicated portions (labeled [##]) with values of appropriate financial variables from the corresponding healthcare system. In another example, a second summary sentence may comprise “Based on the average cost of [##] per hour wasted, the result [of reducing the total amount of hold hours by [##]] is a savings of $[##]K per year” with the logic rules filling the indicated portions (labeled [##]) with values of appropriate financial variables from the corresponding healthcare system.

In some embodiments, the logic rules may prioritize one or more financial variables and/or one or more operating variables, as described above. Such prioritization may allow for use of the selected variables in generating the at least one second summary sentence.

Additional or alternative sentences or clauses may be selected for inclusion in the executive summary. For example, in any of the embodiments described above, the logic rules may further determine a nexus between a first summary sentence and a change in one or more financial variables to select a clause or sentence to append to the first summary sentence. Additionally or alternatively, the logic rules may determine a nexus between a second summary sentence and one or more changes in operating variables to select a clause or sentence to append to the second summary sentence. Additionally or alternatively, the logic rules may determine a synergy between two or more operating variables in order to select a recommendation sentence related to both operating variables (e.g., a number of patients queued for transfer to a radiology department and a number of patients queued for transfer to a surgery department during the same time may combine to produce an amplified negative effect on financial variables). In some embodiments, a model may automatically rank summary sentences and/or variables based on a largest opportunity for improvement (e.g., an improvement toward a financial goal, patient flow minutes, and/or Length of Stay).

The one or more servers or other computing devices may generate an executive summary including the at least one first summary sentence and the at least one second summary sentence. For example, the report may include the sentences with bullet points (as depicted in FIG. 3C) and/or the sentences with one or more corresponding visual indicators (as depicted in FIG. 3D).

The one or more servers or other computing devices may append the generated executive summary to the received report and outputting the report after appending. For example, the one or more servers may output a file encoded in a portable document format (pdf) or the like comprising the appended report.

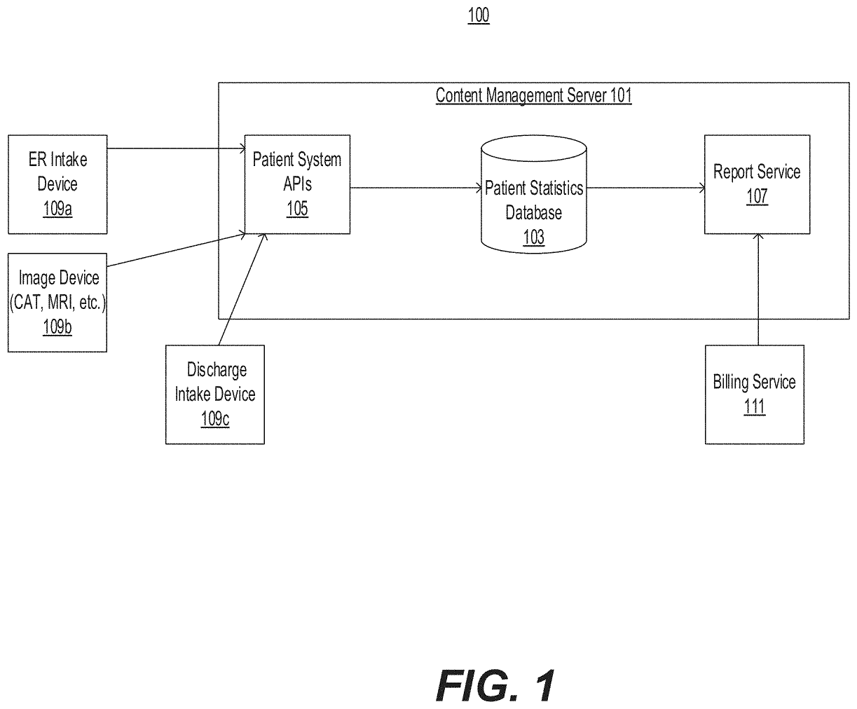

In FIG. 1, there is shown a block diagram of a system 100 for generating a healthcare metric report. As depicted in FIG. 1, system 100 may comprise a server 101 that may receive patient statistics from using one or more APIs, e.g., patient system APIs 105. For example, patient system APIs 105 are depicted as receiving real-time statistics from an emergency room intake device 109a, an imaging device 109b (e.g., an MRI machine, a computed axial tomography (CAT) scanner, or the like), and a discharge intake device 109c. As explained above, additional devices receiving patient-level information may transmit statistics to server 101 via patient system APIs 105. In some embodiments, one or more of patient system APIs 105 may require connection to a private network (and/or to a virtual private network (VPN)) for statistics to be received. This may, for example, allow for health statistics to remain unencrypted without significant risk of interception. Additionally or alternatively, the statistics may be encrypted before sending via patient system APIs 105.

As further depicted in FIG. 1, server 101 may store received health statistics in one or more databases (e.g., patient statistics database 103). In some embodiments, server 101 may aggregate the patient-level statistics before storing and/or after storing. For example

A report service 107 (e.g., on server 101 or on a different system connected securely to server 101) may access the statistics stored in patient statistics database 103 for use in automatically generating a healthcare metric report (e.g., as described above). Additionally, report server 107 may retrieve financial statistics from a billing service 111 for use in automatically generating the healthcare metric report (e.g., as described above).

In some embodiments, report service 107 may prioritize one or more financial variables (e.g., retrieved from billing service 111 or determined from converting operating-level statistics to financial variables using conversion factors). Additionally, report service 107 may further prioritize one or more healthcare operating variables based on the prioritization of the one or more financial variables. Accordingly, report service 107 may generate a report including the financial variable(s) and/or the associated healthcare operating variable(s) displayed in order of the prioritization. Additionally or alternatively, report service 107 may generate a report including a subset financial variable(s) and/or a subset associated healthcare operating variable(s), where the subset is selected based on the prioritization.

In some embodiments, Report service 107 may prioritize the one or more financial variables according to one or more settings input by a user and/or automatically according to a projected impact on at least one of revenue or cost.

In some embodiments, report service 107 may output a report on a periodic basis. The periodic basis may be determined based on a predefined or user-defined time interval, in response to a request received from a user device, or in response to a triggering event such as an exceeded threshold for one or more metrics disclosed herein. In such embodiments, report service 107 may re-prioritize the one or more financial variables (and/or the one or more associated healthcare operating variables) each time a report is output. Accordingly, each report may display the variables in a different order and/or include a different subset of variables each time.

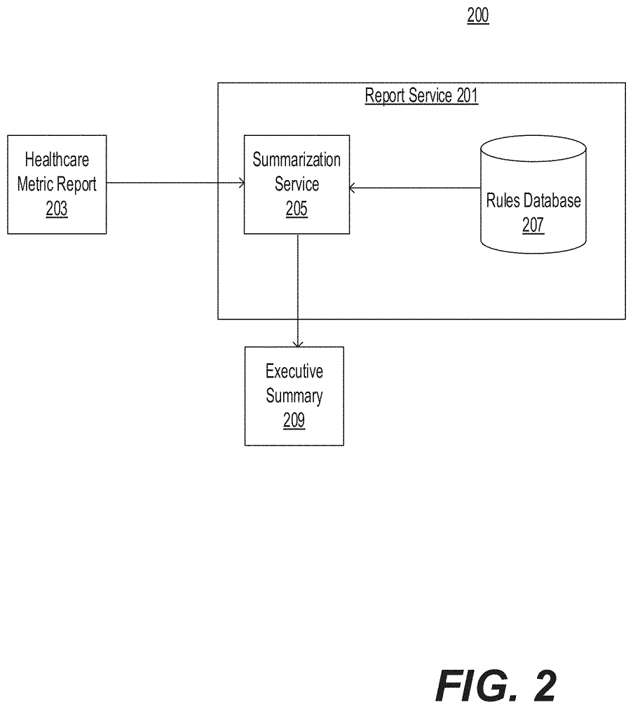

In FIG. 2, there is shown a block diagram of a server 200 including a report service 201 for automatically summarizing a healthcare metric report. For example, server 201 of FIG. 2 may comprise report service 107 of FIG. 1. Accordingly, server 201 of FIG. 2 may comprise at least a portion of server 101 of FIG. 1 and/or may comprise at least a portion of an external system in secure communication with server 101.

As depicted in FIG. 2, report server 201 may comprise a summarization service 205 that receives a healthcare metric report 203. For example, report service 201 may have generated healthcare metric report 203. Alternatively, another report service (e.g., report service 107 of FIG. 1) may have generated healthcare metric report 203. Although not depicted in FIG. 2, summarizing service 205 may receive healthcare metric report 203 using one or more APIs.

As depicted in FIG. 2, the report service 201 may include a rules database 207. For example, rules database 207 may store logic rules that generate summary sentences based on operating variables and/or financial variables and/or recommendation sentences based on summary sentences, operating variables and/or financial variables, as described above. Accordingly, summarization service 205 may fetch appropriate rules from rules database 207 based on operating variables, baselines, and financial variables included in healthcare metric report 203. Summarization service 205 may thus generate executive summary 209 using the logic rules retrieved from rules database 207. As depicted in FIG. 2, summarization service 205 may output executive summary 209 separately. Additionally or alternatively, summarization service 205 may append executive summary 209 to healthcare metric report 203 and output the appended report.

In some embodiments, summarization service 205 may prioritize the one or more financial variables to which the logic rules are applied. Accordingly, determining the at least one second summary sentence may be based on the prioritization. Moreover, summarization service 205 may further prioritize the one or more healthcare operating variables based on the prioritization of the one or more financial variables. Accordingly, determining the at least one first summary sentence may be based on the prioritization of the one or more healthcare operating variables.

Summarization service 205 may prioritize the one or more financial variables according to one or more settings input by a user. For example, as explained above, the settings may comprise a roadmap of operational variables or a direct input of one or more operational variables. Thus, the one or more financial variables corresponding to the operational variables may be prioritized. Additionally or alternatively, summarization service 205 may automatically prioritize the one or more financial variables according to a projected impact on at least one of revenue or cost. Accordingly, the second summary sentences may focus on financial variables with the greatest potential to increase revenue and/or decrease cost. Thus, the first summary sentences may focus on operational variables corresponding to the selected financial variables.

In some embodiments, summarization service 205 may generate the executive summary on a periodic basis, and thus may re-prioritize the one or more financial variables each time an executive summary is generated. The periodic basis may be determined based on a predefined or user-defined time interval, in response to a request received from a user device, or in response to a triggering event such as an exceeded threshold for one or more metrics disclosed herein.

FIG. 3A shows an example graphical user interface (GUI) 300 including an automatically generated healthcare metric report. As depicted in FIG. 3A, one or more visual formats (such as bar graphs, pie charts, or the like) may be generated to represent one or more operating variables as compared to baselines. In the example of FIG. 3A, the baselines may represent goals, e.g., set by the healthcare company and/or input into a report service generating GUI 300. As explained above, the baselines may instead comprise industry averages, medians, or the like.

FIG. 3B shows an example graphical user interface (GUI) 310 including an automatically generated healthcare financial report. As depicted in FIG. 3B, one or more conversion factors (e.g., net per patient contribution margin and operating cost per bed per day) may transform operating variables (e.g., annual admissions, average length of stay, or the like) into financial variables (e.g., revenue per increased admission, cost reduction per day, or the like). Moreover, one or more changes in an operating variable (e.g., length of stay reduction) may transform into a financial variable (e.g., increased revenue from reduced length of stay, reduced operating cost from reduced length of stay) based on the one or more conversion factors. Although depicted using text, GUI 310 may additionally or alternatively include one or more visual formats (such as bar graphs, pie charts, or the like).

FIG. 3C shows an example graphical user interface (GUI) 320 including an automatically generated executive summary. As depicted in FIG. 3C, one or more summary sentences and/or recommendation sentences may be generated based on associated operating variables in a healthcare report, e.g., received by a report service generating GUI 320. In the example of FIG. 3C, one or more operating variables (and/or one or more associated financial variables) indicate that the discrepancy between baseline and discharge time, baseline and waiting time associated with discharge, baseline and bed turnaround time, or the like is large (e.g., above a threshold) and/or that a projected revenue gain and/or operating cost reduction associated with reduction of such operating variables is large (e.g., above a threshold). Although depicted using text, GUI 320 may additionally or alternatively include one or more visual formats (such as bar graphs, pie charts, or the like), such as GUI 330 described below.

FIG. 3D shows another example graphical user interface (GUI) 330 including an automatically generated executive summary. As depicted in FIG. 3D, one or more summary sentences and/or recommendation sentences may be generated based on associated financial variables in a healthcare report, e.g., received by a report service generating GUI 330. In the example of FIG. 3D, one or more financial variables indicate that a projected revenue gain and/or operating cost reduction associated with reduction of such operating variables related to length of stay is large (e.g., above a threshold). Moreover, FIG. 3D visually depicts such projected gains based on the financial variables juxtaposed with associated summary sentences.

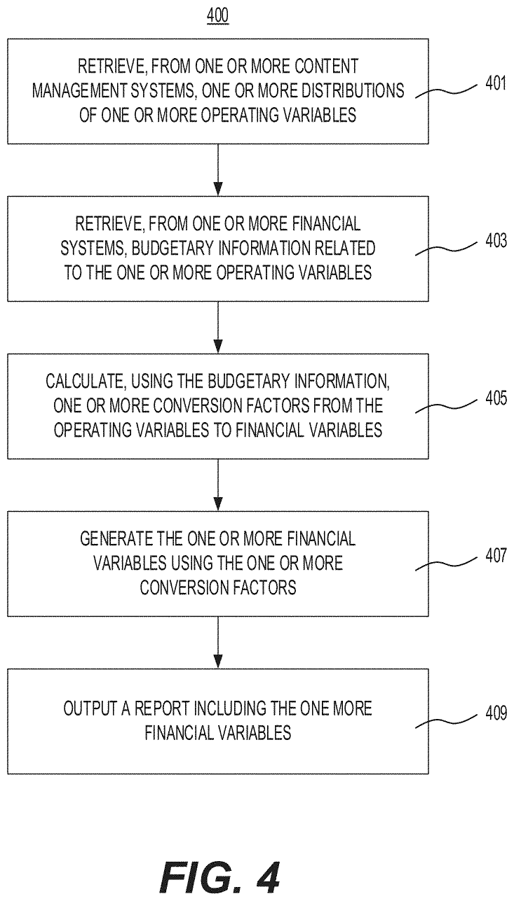

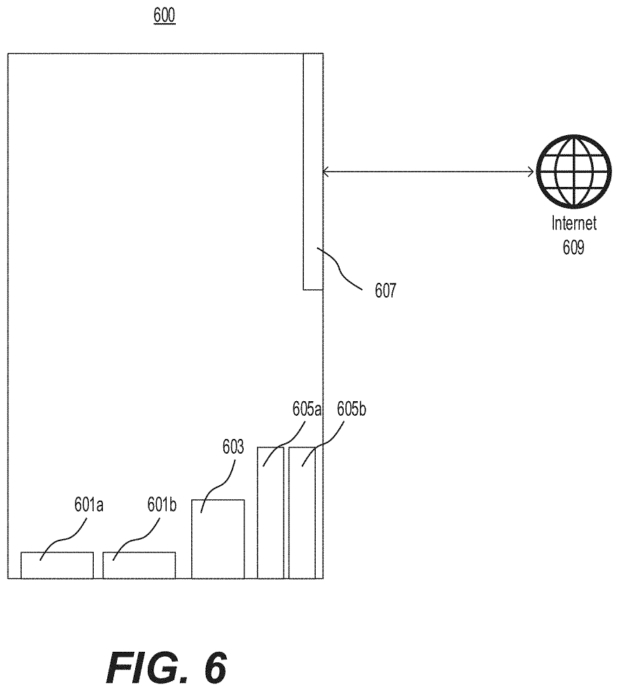

FIG. 4 depicts an example method 400 for automatically generating healthcare metrics. Method 400 may be implemented using one or more processors (e.g., processor 603 of FIG. 6).

At step 401, the processor may retrieve, from one or more networked computer systems, one or more distributions, each distribution associated with a healthcare operating variable, the one or more networked computer systems (e.g., server 101 of FIG. 1) collating the one or more distributions based on real-time patient-by-patient input (e.g., from intake device 109a, imaging device 109b, discharge device 109c, or the like). As explained above, the patient statistics may be sent to the networked computer system(s) over one or more computer networks, such as the Internet, a local area network (LAN), or the like, and may be sent using WiFi, 4G, Ethernet, or the like. In some embodiments, to retain security, the patient statistics may be sent over a private network (such as a LAN) and/or may be encrypted (e.g., using an Advanced Encryption Standard (AES)). The one or more networked computer systems may collate (aggregate) such statistics to generate the distribution(s).

At step 403, the processor may retrieve, from one or more financial systems, budgetary information classified as related to the one or more associated healthcare operating variables. For example, as explained above, a billing system (such as billing service 111 of FIG. 1) may send revenue statistics, operating cost statistics, or the like to the processor. The processor may receive the budgetary information via the same API(s) as the distribution(s) and/or via one or more different APIs.

At step 405, the processor may calculate, using the budgetary information, one or more conversion factors from the one or more associated healthcare operating variables to one or more financial variables. For example, as explained above, the processor may determine a marginal revenue and/or cost associated with the operating variables such that the processor may readily predict changes in revenue and/or cost based on projected changes in the operating variables.

At step 407, the processor may generate the one or more financial variables using the one or more conversion factors. For example, as explained above, the processor may determine revenue and costs associated with particular lengths of stay, on a per-bed basis, on a per-visit basis, associated with particular surgeries, associated with particular visits, associated with particular discharge times, associated with particular transport times, or the like. Additionally or alternatively, the processor may generate the financial variable(s) as projected changes based on projected changes in operating variables. The projected changes in operating variables may be received from a user and/or determined automatically, e.g., by reducing one or more operating variables to an industry-wide (or geographically associated) median, average, or the like.

At step 409, the processor may output a report including the one or more financial variables. For example, the processor may send the report to one or more recipients, e.g., over a secure connection and/or after encrypting of the report. Additionally or alternatively, the processor may send the report to another service (or another part of a report service) for automatic summary generation (e.g., as described below in method 500).

Method 400 may include additional steps. For example, method 400 may further include encoding the report as one or more files using an encoding format (such as pdf or the like). In some embodiments, the encoding may include generating one or more visual representations of the one or more financial variables (and/or of the one or more operating variables compared with baselines), as depicted in FIGS. 3A and 3D. Additionally or alternatively, method 400 may include storing the generated report for later output.

In some embodiments, as described above, the report may include the one or more financial variables in an order according to a prioritization of the variables. Additionally or alternatively, the report may include a subset of the one or more financial variables selected according to the prioritization.

Additionally, the report may include one or more operating variables. In such embodiments, the report may include the one or more operating variables in an order according to a prioritization of the variables. Additionally or alternatively, the report may include a subset of the one or more operating variables selected according to the prioritization. The operating variables may be prioritized according to a corresponding prioritization of financial variables or vice versa.

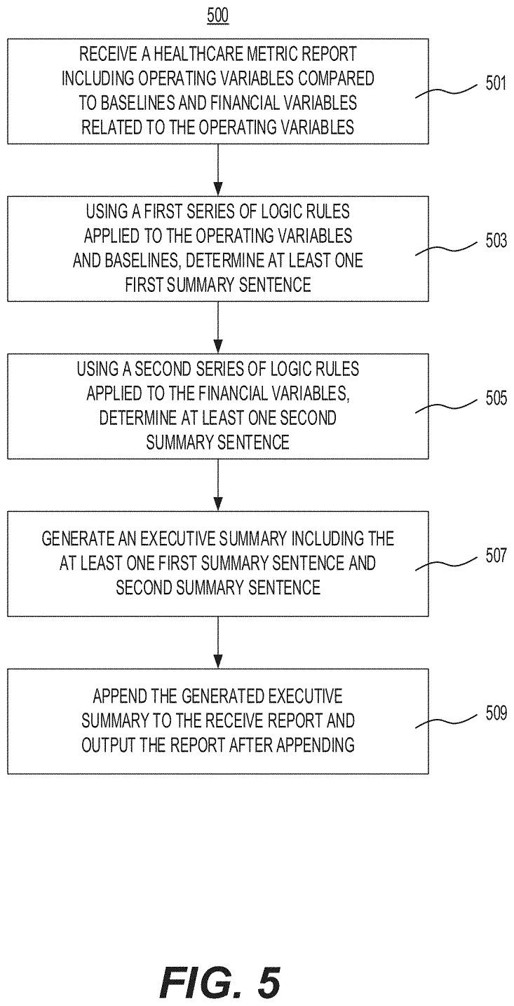

FIG. 5 depicts an example method 500 for automatically summarizing a generated healthcare metric report. Method 500 may be implemented using one or more processors (e.g., processor 603 of FIG. 6). As explained above, method 500 may be executed to summarize a report generated using method 400 of FIG. 4 described above.

At step 501, the processor may receive the healthcare metric report including one or more healthcare operating variables compared to one or more associated baselines and one more financial variables related to the one or more healthcare operating variables. For example, the healthcare metric report may have been generated using method 400 of FIG. 4, described above. In some embodiments, the report may be received over one or more computer networks, such as the Internet, a local area network (LAN), or the like, and may be sent using WiFi, 4G, Ethernet, or the like. In some embodiments, to retain security, the report may be sent over a private network (such as a LAN) and/or may be encrypted (e.g., using an Advanced Encryption Standard (AES)).

At step 503, using a first series of logic rules applied to the one or more healthcare operating variables and associated one or more baselines, the processor may determine at least one first summary sentence. For example, as explained above, the processor may select one or more operating variables having largest discrepancies compared to the associated baseline(s) and may extract one or more predetermined sentences from a rules database (e.g., rules database 207 of FIG. 2) based thereon. In some embodiments, the processor may receive input indicative of systems and protocols employed by a healthcare company associated with the report such that the processor may select the predetermined sentences from a ranked list based on the input.

Additionally or alternatively, as explained above, the processor may prioritize one or more operating variables and generate the at least one first summary sentence accordingly. For example, the first series of logic rules may be configured to generate a predetermined (or user-selected) number of first summary sentences (e.g., one, two, three, four, or the like). Accordingly, the prioritization may allow the first series of logic rules to select sentences corresponding to the top prioritized variables up to the predetermined (or user-selected) number.

At step 505, using a second series of logic rules applied to the one or more financial variables, the processor may determine at least one second summary sentence. For example, as explained above, the processor may select one or more financial variables most likely to be affected by and/or having a largest change based on a change in one or more of the operating variables and may extract one or more predetermined sentences from a rules database (e.g., rules database 207 of FIG. 2) based thereon. In some embodiments, the at least one second summary sentence may include a magnitude of the predicted change(s) in the selected financial variable(s).

Additionally or alternatively, as explained above, the processor may prioritize one or more financial variables and generate the at least one second summary sentence accordingly. For example, the second series of logic rules may be configured to generate a predetermined (or user-selected) number of second summary sentences (e.g., one, two, three, four, or the like). Accordingly, the prioritization may allow the second series of logic rules to select sentences corresponding to the top prioritized variables up to the predetermined (or user-selected) number.

Although described as following step 503, step 505 may precede step 503. For example, the processor may prioritize the one or more financial variables and then prioritize operating variables corresponding to those financial variables, as explained above with respect to FIG. 2.

At step 507, the processor may generate an executive summary including the at least one first summary sentence and the at least one second summary sentence. For example, as explained above, the executive summary may include the sentences using bullet points (as depicted in FIG. 3C) and/or juxtaposed with one or more corresponding visual indicators (as depicted in FIG. 3D) of the selected financial variable(s) (and/or predicted change(s) thereof) and/or of the associated operating variable(s) (and/or the change(s) thereof).

At step 509, the processor may append the generated executive summary to the received report and outputting the report after appending. For example, the processor may send the appended report to one or more recipients, e.g., over a secure connection and/or after encrypting of the appended report.

Method 500 may include additional steps. For example, method 500 may further include encoding the report as one or more files using an encoding format (such as pdf or the like). In some embodiments, the encoding may include generating one or more visual representations to accompany the summary sentences (e.g., displaying one or more related financial variables and/or displaying one or more operating variables compared with baselines), as depicted in FIGS. 3A and 3D. Additionally or alternatively, method 400 may include storing the appended report for later output.

FIG. 6 is block diagram of an example device 600 suitable for implementing the disclosed systems and methods. For example, device 600 may execute method 400 of FIG. 4 and/or method 500 of FIG. 5. Device 600 may comprise a server, desktop computer, or the like. For example, device 600 may comprise server 101 of FIG. 1 or in any other entity configured to generate healthcare metric reports.

As depicted in FIG. 6, example server 600 may include at least one processor (e.g., processor 603) and at least one memory (e.g., memories 605a and 605b).

Processor 603 may comprise a central processing unit (CPU), a graphics processing unit (GPU), or other similar circuitry capable of performing one or more operations on a data stream. Processor 603 may be configured to execute instructions that may, for example, be stored on one or more of memories 605a and 605b.

Memories 605a and 605b may be volatile memory (such as RAM or the like) and/or non-volatile memory (such as flash memory, a hard disk drive, or the like). As explained above, memories 605a and 605b may store instructions for execution by processor 503.

As further depicted in FIG. 6, server 600 may include at least one network interface controller (NIC) (e.g., NIC 607). NIC 607 may be configured to facilitate communication over at least one computing network (e.g., network 609, which is depicted in the example of FIG. 6 as the Internet). Communication functions may thus be facilitated through one or more NICs, which may be wireless and/or wired and may include an Ethernet port, radio frequency receivers and transmitters, and/or optical (e.g., infrared) receivers and transmitters. The specific design and implementation of the one or more NICs depend on the computing network 609 over which server 600 is intended to operate. For example, in some embodiments, server 600 may include one or more wireless and/or wired NICs designed to operate over a GSM network, a GPRS network, an EDGE network, a Wi-Fi or WiMax network, and a Bluetooth® network. Alternatively or concurrently, server 600 may include one or more wireless and/or wired NICs designed to operate over a TCP/IP network.

As depicted in FIG. 6, server 600 may include and/or be operably connected to one or more storage devices, e.g., storages 601a and 601b. Storage devices 601a and 601b may be volatile (such as RAM or the like) or non-volatile (such as flash memory, a hard disk drive, or the like).

Processor 603, memories 605a and 605b, NIC 607, and/or storage devices 601a and 601b may comprise separate components or may be integrated in one or more integrated circuits. The various components in server 600 may be coupled by one or more communication buses or signal lines (not shown).

FIG. 7 is block diagram of an example intake device 700 for collecting patient data for inclusion in a networked computer system (e.g., server 101 of FIG. 1, which may comprise server 600 of FIG. 6). Device 700 may comprise a smartphone, a tablet, a wearable like a Fitbit®, or the like.

Device 700 may have a screen 701. For example, screen 701 may display one or more graphical user interfaces (GUIs). In certain aspects, screen 701 may comprise a touchscreen to facilitate use of the one or more GUIs.

As further depicted in FIG. 7, intake device 700 may have at least one processor 703. For example, at least one processor 703 may comprise a system-on-a-chip (SOC) adapted for use in a portable device, such as device 700. Alternatively or concurrently, at least one processor 703 may comprise any other type(s) of processor.

As further depicted in FIG. 7, intake device 700 may have one or more memories, e.g., memories 705a and 705b. In certain aspects, some of the one or more memories, e.g., memory 705a, may comprise a volatile memory. In such aspects, memory 705a, for example, may store one or more applications (or “apps”) for execution on at least one processor 703. For example, an app may include an operating system for intake device 700 and/or an app for collecting data and providing an application programming interface (API) to one or more authorized networked computer systems (e.g., server 101 of FIG. 1). In addition, memory 705a may store data generated by, associated with, or otherwise unrelated to an app in memory 705a.

Alternatively or concurrently, some of the one or more memories, e.g., memory 705b, may comprise a non-volatile memory. In such aspects, memory 705b, for example, may store one or more applications (or “apps”) for execution on at least one processor 703. For example, as discussed above, an app may include an operating system for intake device 700 and/or an app for collecting healthcare data and providing the data to authorized parties via one or more APIs. In addition, memory 705b may store data generated by, associated with, or otherwise unrelated to an app in memory 705b. Furthermore, memory 705b may include a pagefile, swap partition, or other allocation of storage to allow for the use of memory 705b as a substitute for a volatile memory if, for example, memory 705a is full or nearing capacity.

As depicted in FIG. 7, intake device 700 may include at least one network interface controller (NIC) (e.g., NIC 707). NIC 707 may be configured to facilitate communication over at least one computing network. Communication functions may thus be facilitated through one or more NICs. Although depicted in wireless in FIG. 7 and including radio frequency receivers and transmitters and/or optical (e.g., infrared) receivers and transmitters, NIC 707 may alternatively be wired and include an Ethernet port or the like. The specific design and implementation of the one or more NICs depend on the computing network over which intake device 700 is intended to operate. For example, in some embodiments, intake device 700 may include one or more wireless and/or wired NICs designed to operate over a GSM network, a GPRS network, an EDGE network, a Wi-Fi or WiMax network, and a Bluetooth® network. Alternatively or concurrently, intake device 700 may include one or more wireless and/or wired NICs designed to operate over a TCP/IP network.

Each of the above identified instructions and applications may correspond to a set of instructions for performing one or more functions described above. These instructions need not be implemented as separate software programs, procedures, or modules. Disclosed memories may include additional instructions or fewer instructions. Furthermore, device 700 may securely deliver patient statistics to server 500 (which may, for example, comprise server 101 of FIG. 1). For example, device 700 may send a patient statistic to server 500, and server 500 may store the update for later inclusion in a report, e.g., generated using method 400 of FIG. 4. These functions of device 700 may be implemented in hardware and/or in software, including in one or more signal processing and/or application specific integrated circuits.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. For example, the described implementations include hardware and software, but systems and methods consistent with the disclosed embodiments can be implemented with hardware alone. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations and/or alterations based on the disclosed embodiments. The elements in the claims are to be interpreted broadly based on the language employed in the claims and not limited to examples described in the present specification or during the prosecution of the application, which examples are to be construed as nonexclusive.

Instructions or operational steps stored by a computer-readable medium may be in the form of computer programs, program modules, or codes. As described herein, computer programs, program modules, and code based on the written description of this specification, such as those used by the processor, are readily within the purview of a software developer. The computer programs, program modules, or code can be created using a variety of programming techniques. For example, they can be designed in or by means of Java, C, C++, assembly language, or any such programming languages. One or more of such programs, modules, or code can be integrated into a device system or existing communications software. The programs, modules, or code can also be implemented or replicated as firmware or circuit logic.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A system for automatically generating healthcare metrics, the system comprising:
at least one memory storing instructions; and
at least one processor configured to execute the instructions to:
retrieve, from one or more networked computer systems, one or more distributions, each distribution associated with a healthcare operating variable, the one or more networked computer systems collating the one or more distributions based on real-time patient-by-patient input;
retrieve, from one or more financial systems, budgetary information classified as related to the one or more associated healthcare operating variables;
calculate, using the budgetary information, one or more conversion factors from the one or more associated healthcare operating variables to one or more financial variables;
generate the one or more financial variables using the one or more conversion factors; and
output a report including the one or more financial variables.

2. The system of claim 1, wherein the at least one processor is further configured to prioritize the one or more financial variables.

3. The system of claim 2, wherein the at least one processor is further configured to prioritize the one or more associated healthcare operating variables based on the prioritization of the one or more financial variables, and wherein the report further includes the one or more associated healthcare operating variables.

4. The system of claim 2, wherein the at least one processor is configured to prioritize the one or more financial variables according to one or more settings input by a user.

5. The system of claim 2, wherein the at least one processor is configured to automatically prioritize the one or more financial variables according to a projected impact on at least one of revenue or cost.

6. The system of claim 2, wherein the at least one processor is configured to output the report on a periodic basis, and re-prioritize the one or more financial variables each time a report is output.

7. A system for automatically summarizing a generated healthcare metric report, the system comprising:
at least one memory storing instructions; and
at least one processor configured to execute the instructions to:
receive the healthcare metric report including one or more healthcare operating variables compared to one or more associated baselines and one more financial variables related to the one or more healthcare operating variables;
using a first series of logic rules applied to the one or more healthcare operating variables and associated one or more baselines, determine at least one first summary sentence;
using a second series of logic rules applied to the one or more financial variables, determine at least one second summary sentence;
generate an executive summary including the at least one first summary sentence and the at least one second summary sentence; and
append the generated executive summary to the received report and outputting the report after appending.

8. The system of claim 7, wherein the at least one processor is further configured to prioritize the one or more financial variables, and wherein determining the at least one second summary sentence is based on the prioritization.

9. The system of claim 8, wherein the at least one processor is further configured to prioritize the one or more healthcare operating variables based on the prioritization of the one or more financial variables, and wherein determining the at least one first summary sentence is based on the prioritization of the one or more healthcare operating variables.

10. The system of claim 8, wherein the at least one processor is configured to prioritize the one or more financial variables according to one or more settings input by a user.

11. The system of claim 8, wherein the at least one processor is configured to automatically prioritize the one or more financial variables according to a projected impact on at least one of revenue or cost.

12. The system of claim 8, wherein the at least one processor is configured to generate the executive summary on a periodic basis, and re-prioritize the one or more financial variables each time an executive summary is generated.

13. A method for automatically generating healthcare metrics, comprising:
retrieving, from one or more networked computer systems, one or more distributions, each distribution associated with a healthcare operating variable, the one or more networked computer systems collating the one or more distributions based on real-time patient-by-patient input;
retrieving, from one or more financial systems, budgetary information classified as related to the one or more associated healthcare operating variables;
calculating, using the budgetary information, one or more conversion factors from the one or more associated healthcare operating variables to one or more financial variables;
generating the one or more financial variables using the one or more conversion factors; and
outputting a report including the one or more financial variables.

14. The method of claim 13 further comprising prioritizing the one or more financial variables.

15. The method of claim 14 further comprising prioritizing the one or more associated healthcare operating variables based on the prioritization of the one or more financial variables, and wherein the report further includes the one or more associated healthcare operating variables.

16. The method of claim 14 further comprising prioritizing the one or more financial variables according to one or more settings input by a user.

17. The method of claim 14 further comprising automatically prioritizing the one or more financial variables according to a projected impact on at least one of revenue or cost.

18. The method of claim 14, wherein the report is output on a periodic basis, and the method further comprises re-prioritizing the one or more financial variables each time a report is output.
